(19) United States
(12) Patent Application Publication
(10) Pub. No.: US 2023/0288553 A1
(43) Pub. Date: Sep. 14, 2023

(54) MEDICAL CARE RADAR SYSTEM

(71) Applicant: RAYINN TECHNOLOGY, INC., Hsinchu (TW)

(72) Inventors: Shih-Hai TU, Hsinchu (TW); Ming-Fa TSAI, Hsinchu (TW)

(73) Assignee: RAYINN TECHNOLOGY, INC., Hsinchu (TW)

(21) Appl. No.: 17/725,128

(22) Filed: Apr. 20, 2022

(30) Foreign Application Priority Data
Mar. 11, 2022 (TW) 111109004

(51) Int. Cl. G01S 13/87; G01S 13/88; G01S 7/282; G01S 7/35; A61B 5/05

(52) U.S. Cl. CPC G01S 13/872; G01S 13/88; G01S 7/282; G01S 7/35; A61B 5/05

(57) ABSTRACT

Disclosed is a medical care radar system, comprising: a radio-frequency integrated circuit, a first-operation-mode transmitting antenna, a second-operation-mode transmitting antenna, a first-operation-mode receiving antenna set, a second-operation-mode receiving antenna set, a processing device, and an analog-to-digital converter. A field-programmable gate array of the processing device controls the analog-to-digital converter to perform an analog-to-digital conversion without the use of a microcontroller, so that the architecture is simplified and the cost is reduced.

### FIELD OF THE INVENTION

The present invention relates to a physiological sensing device, and more particularly relates to a medical care radar system.

### BACKGROUND OF THE INVENTION

A conventional physiological sensing device, when detecting two kinds of physiological signals, is generally provided with two one-transmitter-two-receiver modules, i.e., two 1T2R radar modules as shown in FIG. 1. And there are two radio-frequency integrated circuits 10 provided for this architecture correspondingly. Each 1T2R radar module firstly converts the analog signal received by the radar into a digital signal by utilizing an analog-to-digital converter (ADC) 70 together with a microcontroller (MCU) 710, and then a field-programmable gate array and a central processor unit of a processing device 60 perform calculating operation, such as Fourier transform, on the digital signal. Such architecture, which is provided with two analog-to-digital converters 70 and two microcontrollers 710, is complicated and costly.

### SUMMARY OF THE INVENTION

Accordingly, one objective of the present invention is to provide a medical care radar system, which has simplified architecture and lower cost.

In order to overcome the technical problems in prior art, the present invention provides a medical care radar system, comprising: one radio frequency integrated circuit which is operated in a first operation mode and a second operation mode; a first-operation-mode transmitting antenna, which is connected to the radio frequency integrated circuit, wherein when the radio frequency integrated circuit is operated in the first operation mode, the radio frequency integrated circuit emits a first signal through the first-operation-mode transmitting antenna; a second-operation-mode transmitting antenna, which is connected to the radio frequency integrated circuit, wherein when the radio frequency integrated circuit is operated in the second operation mode, the radio frequency integrated circuit emits a second signal through the second-operation-mode transmitting antenna; a first-operation-mode receiving antenna set, which is connected to the radio frequency integrated circuit, wherein the first-operation-mode receiving antenna set receives a first reflected signal, the first reflected signal is a reflected signal of the first signal reflected from a human body; a second-operation-mode receiving antenna set, which is connected to the radio frequency integrated circuit, and the second-operation-mode receiving antenna set receiving a second reflected signal, the second reflected signal is a reflect signal of the second signal reflected from the human body; a processing device having a field-programmable gate array and a central processing unit which is connected to the field-programmable gate array; and an analog-to-digital converter, which is connected between the radio frequency integrated circuit and the field-programmable gate array, wherein the radio frequency integrated circuit generates a sensing data signal according to the first reflected signal and the second reflected signal, the analog-to-digital converter performs an analog-to-digital conversion on the sensing data signal to obtain an digital sensing data signal according to a conversion controlling signal transmitted from the field-programmable gate array to the analog-to-digital converter, and the analog-to-digital converter transmits the digital sensing data signal to the field-programmable gate array, and the field-programmable gate array transmits the digital sensing data signal to the central processing unit such that the central processing unit performs a baseband signal operation on the digital sensing data signal and output a sensing result.

In one embodiment of the present invention, the first signal is a pulse signal.

In one embodiment of the present invention, the second signal is a frequency modulated continuous wave signal.

In one embodiment of the present invention, the medical care radar system further comprises a power amplifier, which is connected between the first-operation-mode transmitting antenna and the radio frequency integrated circuit.

In one embodiment of the present invention, the radio frequency integrated circuit operates between the first operation mode and the second operation mode in a time division manner.

In one embodiment of the present invention, the medical care radar system further comprises a human-machine interface, which is connected to the processing device, wherein the human-machine interface has an input device to which a user input commands to configure the processing device.

In one embodiment of the present invention, the human-machine interface has a display screen, and the human-machine interface receives the sensing result and displays the sensing result on the display screen.

With the technical means adopted by the present invention, the present invention integrates two 1T2R radar modules by sharing the same analog digital converter, and the analog-to-digital conversion operated by the microcontroller of the analog digital converter in the prior art is performed by a field-programmable gate array. Since the field-programmable gate array replaces the functionality of the microcontroller, the microcontroller is omitted so that the architecture is simplified and the cost is reduced. In addition, different from the architecture that the functionality of the microcontroller is achieved by utilizing software, the field-programmable gate array utilizes hardware circuits to process the analog-to-digital conversion, which reduces the signal calculation load on the central processing unit and enables faster radar signal processing.

### BRIEF DESCRIPTION OF THE DRAWINGS

FIG. 1 is a schematic block diagram illustrating a conventional physiological sensing device;

FIG. 2 is a schematic block diagram illustrating a medical care radar system according to an embodiment of the present invention;

FIG. 3 is a schematic block diagram illustrating a part of the medical care radar system according to an embodiment of the present invention;

FIG. 4 is a schematic waveform diagram illustrating a conversion controlling signal in the medical care radar system according to the embodiment of the present invention; and

FIG. 5 is a schematic state diagram illustrating an analog-to-digital convertor of the medical care radar system according to the embodiment of the present invention.

### DETAILED DESCRIPTION OF THE PREFERRED EMBODIMENTS

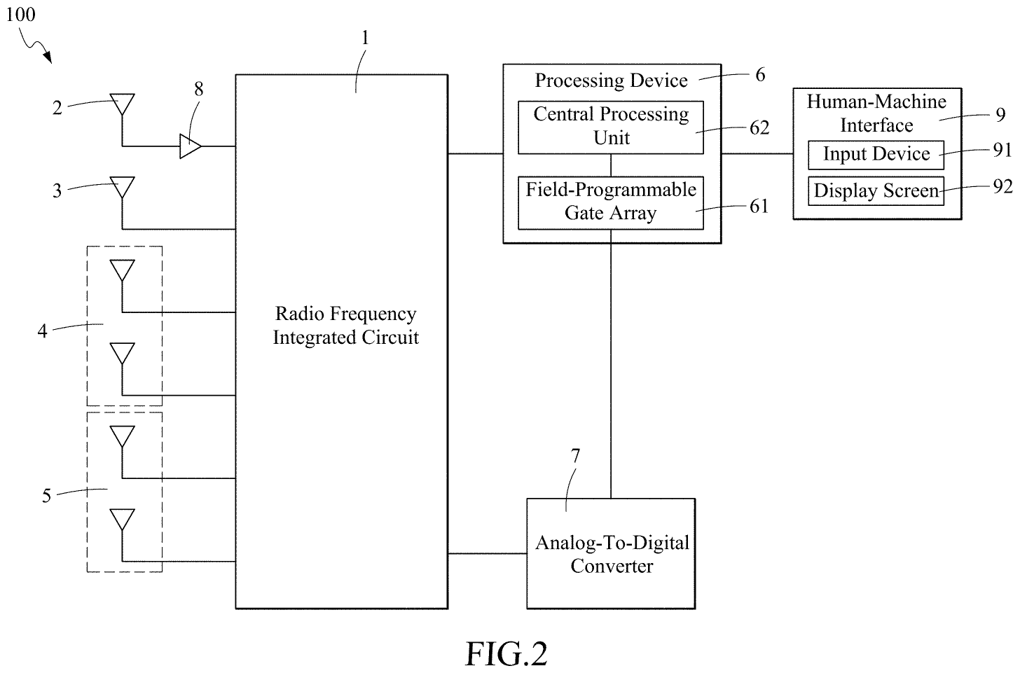

The preferred embodiments of the present invention are described in detail below with reference to FIG. 2 to FIG. 5. The description is used for explaining the embodiments of the present invention only, but not for limiting the scope of the claims.

As shown in FIG. 2 to FIG. 4, a medical care radar system 100 according to one embodiment of the present invention includes: one radio frequency integrated circuit (RFIC) 1, a first-operation-mode transmitting antenna 2, a second-operation-mode transmitting antenna 3, a first-operation-mode receiving antenna set 4, a second-operation-mode receiving antenna set 5, a processing device 6, and an analog-to-digital converter (ADC) 7.

As shown in FIG. 2, the radio frequency integrated circuit 1 is connected to the first-operation-mode transmitting antenna 2, the second-operation-mode transmitting antenna 3, the first-operation-mode receiving antenna set 4, and the second-operation-mode receiving antenna set 5. In this embodiment, the first-operation-mode transmitting antenna 2 and the second-operation-mode transmitting antenna 3 each have one transmitting antenna. The first-operation-mode receiving antenna set 4 and the second-operation-mode receiving antenna set 5 each have two receiving antennas. In other words, the medical care radar system 100 of the present invention is a 2T4R architecture with two transmitting antennas and four receiving antennas. It goes without saying that the present invention is not limited to this. In other embodiments, the medical care radar system can be a 3T4R architecture or other architecture with at least two transmitting antennas and at least four receiving antennas.

The radio frequency integrated circuit 1 is responsible for functions of a modulator/demodulator (modem), a switch, a low noise amplifier (LNA), and a filter for example. The radio frequency integrated circuit 1 is operable in a first operation mode and a second operation mode, and thus provides functions of at least two types of radar in combination. In other embodiments, the radio frequency integrated circuit 1 also can be operated in a third operation mode or more modes. Accordingly, the medical care radar system would have transmitting antennas and receiving antennas corresponding to the said modes.

In this embodiment, the first operation mode and the second operation mode are used for detecting heartbeat and detecting human body motion, respectively. Accordingly, the first signal is a pulse signal, and the second signal is a frequency modulated continuous wave (FMCW) signal. Depending on different physiological signals to be sensed, the first signal and the second signal can also be other corresponding signals.

When the radio frequency integrated circuit 1 is operated in the first operation mode, the radio frequency integrated circuit 1 emits a first signal through the first-operation-mode transmitting antenna 2. The wavelength of the first signal is within the range of mmWave.

The first-operation-mode receiving antenna set 4 is connected to the radio frequency integrated circuit 1. The radio frequency integrated circuit 1 receives a first reflected signal through the first-operation-mode receiving antenna set 4, wherein the first reflected signal is a reflected signal of the first signal reflected from a human body.

A power amplifier 8 is connected between the first-operation-mode transmitting antenna 2 and the radio frequency integrated circuit 1 to amplify the power of the first signal to ensure that the first reflected signal is sufficient for detecting the human heartbeat. In the case of sensing other physiological signals, the power amplifier 8 may not be provided.

When the radio frequency integrated circuit 1 is operated in the second operation mode, the radio frequency integrated circuit 1 emits a second signal through the second-operation-mode transmitting antenna 3. The wavelength of the second signal is within the range of mmWave.

The second-operation-mode receiving antenna set 5 is connected to the radio frequency integrated circuit 1. The radio frequency integrated circuit 1 receives a second reflected signal through the second-operation-mode receiving antenna set 5, wherein the second reflected signal is a reflected signal of the second signal reflected from the human body. The second-operation-mode receiving antenna set 5 is not limited to operating during the second operation mode, but can also operate during the first operation mode to receive the second signal reflected.

In detail, the first-operation-mode receiving antenna set 4 and the second-operation-mode receiving antenna set 5 each have a slightly difference between the two receiving antennas thereof. For the heartbeat of the human body, the heartbeat can be detected by using the slight difference in position and the micro-Doppler effect. Through filtering extraction, the frequency offset over time of the reflected pulse signal relative to the pulse signal can be extracted, and accordingly the heartbeat of the human body can be detected. The detection range is 30˜200 heartbeats per minute.

For the movement of the human body, since different movements of the human body will generate different micro-Doppler signatures, the movement of the human body, such as lying down, walking, can be detected by utilizing the aforementioned slight difference in position and judging what kind of micro-Doppler signatures the sensing data signal belongs to.

The radio frequency integrated circuit 1 may operate between the first operation mode and the second operation mode in a time division manner, or may operate simultaneously or operate with overlapping part of the operation time.

As shown in FIG. 2, the processing device 6 is a system on a chip (SoC) that has a field-programmable gate array (FPGA) 61 and a central processing unit 62, which is responsible for baseband signal processing before modulation and after demodulation. The central processing unit 62 is connected to the field-programmable gate array 61.

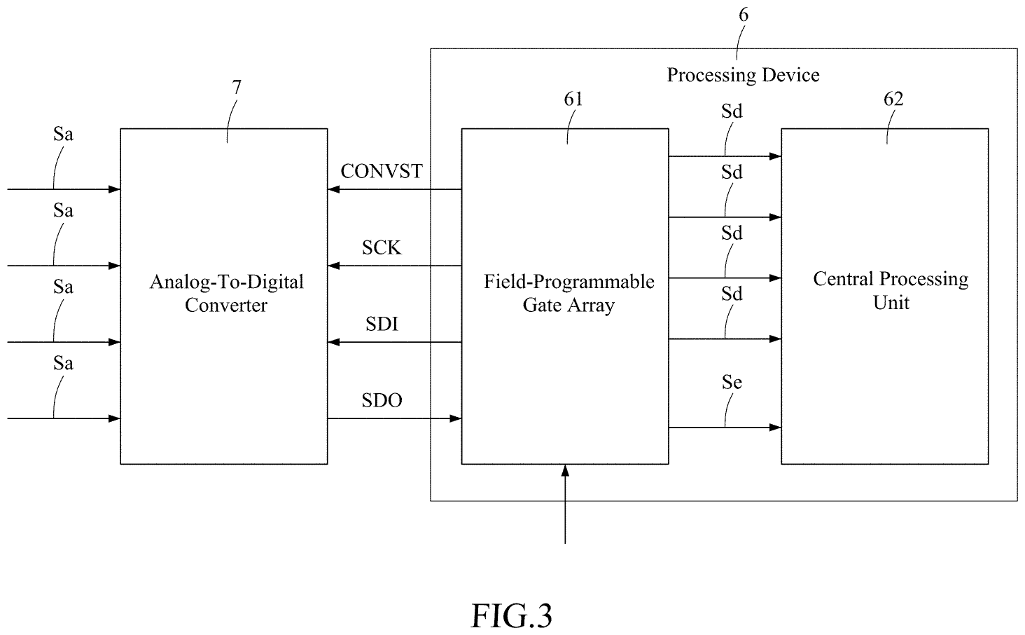

As shown in FIG. 2 and FIG. 3, the analog-to-digital converter 7 is connected between the radio frequency integrated circuit 1 and the field-programmable gate array 61. The radio frequency integrated circuit 1 generates a sensing data signal Sa according to the first reflected signal and the second reflected signal.

As shown in FIG. 3 to FIG. 5, the analog-to-digital converter 7 performs an analog-to-digital conversion on the sensing data signal Sa to obtain an digital sensing data signal Sd according to a conversion controlling signal transmitted from the field-programmable gate array 61 to the analog-to-digital converter 7, and the analog-to-digital converter 7 transmits the digital sensing data signal Sd to the field-programmable gate array 61. In detail, the analog-to-digital converter 7 is a chip with a serial peripheral interface (SPI). The conversion controlling signal received by the analog-to-digital converter 7 includes a conversion start (CONVST) signal, a serial data input (SDI) signal and a serial data clock (SCK) signal. A serial data output (SDO) signal is the digital sensing data signal Sd. It goes without say that depending on the analog digital converter 7 used, the received conversion controlling signal changes accordingly.

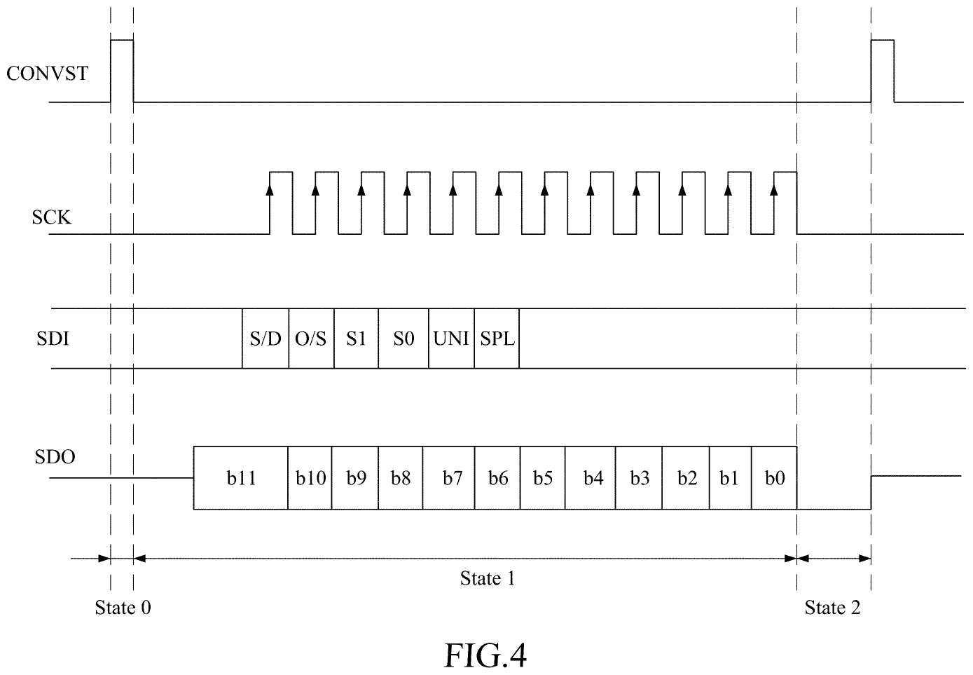

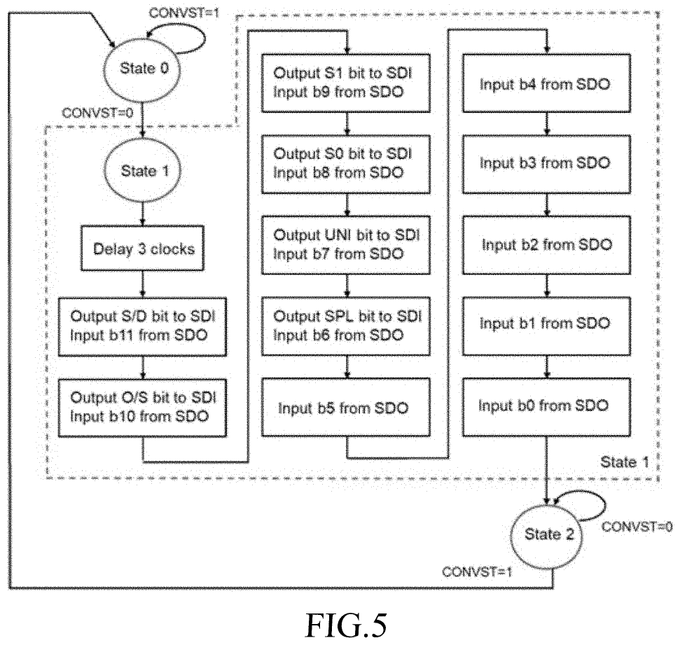

In this embodiment, the analog-to-digital converter 7 is a multiplexed 12-bit ADC. As shown in FIG. 4 and FIG. 5, the conversion start signal controls the analog-to-digital converter 7 to initiate the analog-to-digital conversion process. The serial data input signal controls a multiplexer of the analog-to-digital converter 7 to select a specific input. The serial data output signal is a 12-bit digital sensing data signal Sd. It goes without say that the number of bits of the digital sensing data signal Sd may be other than 12.

Since the medical care radar system 100 of the present invention uses the field-programmable gate array 61 to process the analog-to-digital conversion, the field-programmable gate array 61 replaces the functionality of the conventional microcontroller 710 and therefore the microcontroller 710 is omitted, so that the architecture is simplified and the cost is reduced. In addition, different from the architecture that the functionality of the microcontroller 710 is achieved by utilizing software, the field-programmable gate array 61 utilizes hardware circuits to process analog-to-digital conversion, which reduces the signal calculation load on the central processing unit 62 and enables faster radar signal processing.

After the field-programmable gate array 61 reads the digital sensing data signal Sd, the digital sensing data signal Sd is sent to the register of the central processing unit 62. And the field-programmable gate array 61 will send an external interrupt signal Se to notify the central processing unit 62 to read the converted digital sensing data signal Sd from the register address such that the central processing unit performs operations such as signal processing and a baseband signal operation to output a sensing result. The sensing result includes a heart rate and motion of a sensed subject.

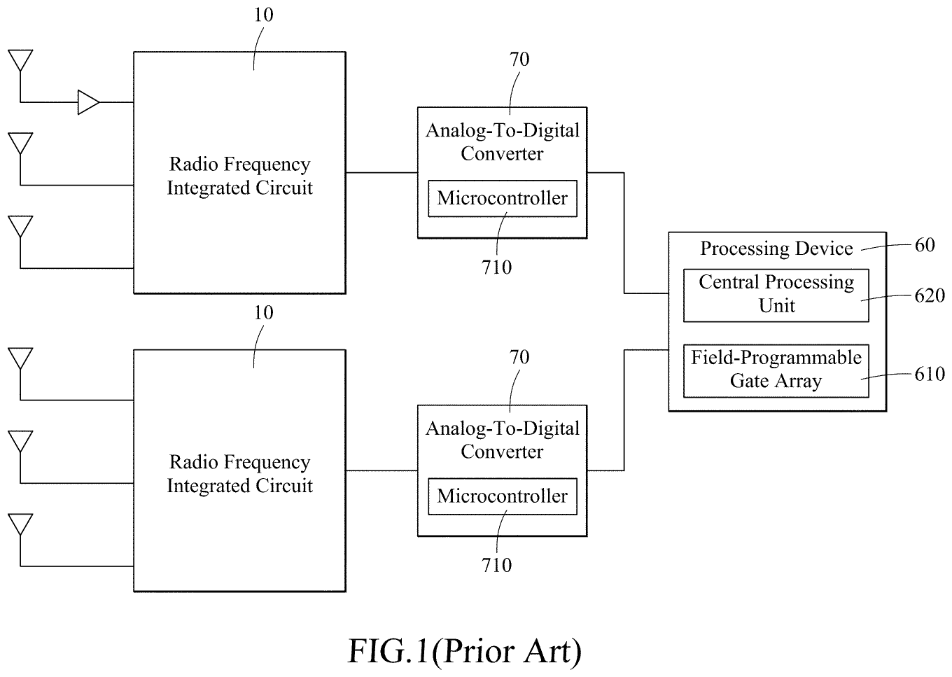

As shown in FIG. 1, in the medical care radar system 100 according to the embodiment of the present invention, a human-machine interface 9 is connected to the processing device 6. The human-machine interface 9 has an input device 91 to which a user input commands to configure the processing device 6. The human-machine interface 9 has a display screen 92, and the human-machine interface 9 receives the sensing result and displays the sensing result on the display screen 92. In other embodiments, the processing device 6 outputs the sensing result to a control center.

The above description should be considered as only the discussion of the preferred embodiments of the present invention. However, a person having ordinary skill in the art may make various modifications without deviating from the present invention. Those modifications still fall within the scope of the present invention.

What is claimed is:

1. A medical care radar system, comprising:
one radio frequency integrated circuit which is operated in a first operation mode and a second operation mode;
a first-operation-mode transmitting antenna, which is connected to the radio frequency integrated circuit, wherein when the radio frequency integrated circuit is operated in the first operation mode, the radio frequency integrated circuit emits a first signal through the first-operation-mode transmitting antenna;
a second-operation-mode transmitting antenna, which is connected to the radio frequency integrated circuit, wherein when the radio frequency integrated circuit is operated in the second operation mode, the radio frequency integrated circuit emits a second signal through the second-operation-mode transmitting antenna;
a first-operation-mode receiving antenna set, which is connected to the radio frequency integrated circuit, wherein the radio frequency integrated circuit receives a first reflected signal through the first operation mode receiving antenna set, the first reflected signal is a reflected signal of the first signal reflected from a human body;
a second-operation-mode receiving antenna set, which is connected to the radio frequency integrated circuit, wherein the radio frequency integrated circuit receives a second reflected signal through the second-operation-mode receiving antenna set, the second reflected signal is a reflect signal of the second signal reflected from the human body;
a processing device having a field-programmable gate array and a central processing unit which is connected to the field-programmable gate array; and
an analog-to-digital converter, which is connected between the radio frequency integrated circuit and the field-programmable gate array,
wherein the radio frequency integrated circuit generates a sensing data signal according to the first reflected signal and the second reflected signal, the analog-to-digital converter performs an analog-to-digital conversion on the sensing data signal to obtain an digital sensing data signal according to a conversion controlling signal transmitted from the field-programmable gate array to the analog-to-digital converter, and the analog-to-digital converter transmits the digital sensing data signal to the field-programmable gate array, and the field-programmable gate array transmits the digital sensing data signal to the central processing unit such that the central processing unit performs a baseband signal operation on the digital sensing data signal and output a sensing result.

2. The medical care radar system of claim 1, wherein the first signal is a pulse signal.

3. The medical care radar system of claim 1, wherein the second signal is a frequency modulated continuous wave signal.

4. The medical care radar system of claim 1, further comprising a power amplifier, which is connected between the first-operation-mode transmitting antenna and the radio frequency integrated circuit.

5. The medical care radar system of claim 1, wherein the radio frequency integrated circuit operates between the first operation mode and the second operation mode in a time division manner.

6. The medical care radar system of claim 1, further comprising a human-machine interface, which is connected to the processing device, wherein the human-machine interface has an input device to which a user input commands to configure the processing device.

7. The medical care radar system of claim 6, wherein the human-machine interface has a display screen, and the human-machine interface receives the sensing result and displays the sensing result on the display screen.
